(19) United States
(12) Patent Application Publication
(10) Pub. No.: US 2017/0312537 A1
(43) Pub. Date: Nov. 2, 2017

(54) PHOTODYNAMIC THERAPY DEVICE

(71) Applicant: Sharp Kabushiki Kaisha, Sakai City, Osaka (JP)

(72) Inventors: Jun MORI, Sakai City (JP); Hidenori KAWANISHI, Sakai City (JP)

(21) Appl. No.: 15/520,441

(22) PCT Filed: Oct. 5, 2015
(86) PCT No.: PCT/JP2015/078249

§ 371 Date: Apr. 20, 2017

(30) Foreign Application Priority Data
Nov. 19, 2014 (JP) 2014-234462

(51) Int. Cl. A61N 5/06; H01L 51/50

(52) U.S. Cl. CPC A61N 5/062; A61N 5/0601; A61N 5/06; H01L 51/50; A61N 2005/0652

(57) ABSTRACT

A light source (2) including a plurality of LEDs (4); a light detector (3) that detects intensity of light emitted by the plurality of LEDs (4) as light intensity distribution of light emitted by the light source (2); and a light intensity distribution control circuit (6) that controls current, by which each of the plurality of LEDs (4) is driven, such that the intensity of the light emitted by each of the plurality of the LEDs (4), which is detected by the light detector (3), falls within a predetermined range.

### TECHNICAL FIELD

The present invention relates to a photodynamic therapy device that treats an affected area by exciting a photosensitive substance that is administered and retained in a patient through radiation of light of a specific wavelength.

### BACKGROUND ART

Photo Dynamic Therapy (PDT) is a method of treatment in which active oxygen or the like is generated by a chemical reaction that arises when a photosensitive substance with an affinity for abnormal cells or a tumor is irradiated with light of a specific wavelength and the abnormal cells or the tumor are necrotized by a bactericidal activity of the active oxygen. Much attention has been recently drawn to the PDT from a viewpoint of QOL (Quality Of Life) because it does not damage normal cells.

Meanwhile, laser is mainly used as a light source used for the PDT. A reason therefor is, for example, as follows: the laser emits monochromatic light and is able to effectively excite a photosensitive substance having a narrow absorption band; the laser has high light intensity density; and the laser is able to generate pulse light. However, laser light is normally spot light, has a narrow radiation coverage, and hence is not suitable for treatment of skin disease or the like.

In recent years, a group of Professor Daisuke Tsuruta, Instructor Toshiyuki Ozawa, et al. of Graduate School of Medicine, Osaka City University has published the first success in the world in treatment of a Methicillin-resistant Staphylococcus aureus (MRSA) infected skin ulcer by conducting systemic administration of 5-aminolevulinic acid (ALA) that is natural amino acid and the PDT with the use of LED (Light Emitting Diode) light with a wavelength of 410 nm (refer to NPL 1).

The ALA is a precursor of a porphyrin-based compound in a heme biosynthetic pathway, and does not have photosensitizing properties. When a given amount of hemes are produced, physiologically, biosynthesis of the ALA is inhibited by a negative feedback mechanism. However, when exogenous ALA is excessively administered, the negative feedback mechanism is invalid, ferrochelatase that is a rate limiting enzyme in heme biosynthesis is depleted, and a large amount of biologically-inherent porphyrin-based compounds, particularly, protoporphyrin IX (hereinafter, described as “PpIX”) are accumulated in cells. In the PDT with the use of the ALA, the PpIX is used as a photosensitizing substance. Such a method of treatment does not cause new resistant bacteria, and is hence expected as a new method of treating bacterial infection in the modern medicine in which there is difficulty in treatment of resistant bacteria.

Regarding the technique as described above, some PDT devices using LEDs are introduced in NPL 2, but are not typical in Japan. A factor thereof is considered as follows: a halogen lamp, a xenon lamp, or a metal halide lamp is generally used in a PDT device. In particular, it is considered that there is no LED light source that covers a wavelength region of 410 nm. Each of the lamps described above has low light emission efficiency and generates a large amount of heat. Thus, a PDT device that uses LEDs having high light emission efficiency is expected.

PTL 1 proposes alternative PDT methods using ALA that are free from side effects (e.g., pain) but have high therapeutic efficacy. PTL 1 describes that the PDT using the ALA causes a side effect of photosensitivity and involves pain making the therapy unacceptable depending on light intensity. According to literatures introduced in PTL 1, it is considered to be implied that the aforementioned side effect occurs when the light intensity is at a certain level or more.

PTL 2 discloses a PDT device that includes a plurality of light source units each of which is constituted by a light source, a sensor, a multi-reflecting member, a condensing lens, and a projection lens.

### CITATION LIST

### Patent Literature

PTL 1: Japanese Unexamined Patent Application Publication No. 2014-94963 (published on May 22, 2014)

PTL 2: Japanese Unexamined Patent Application Publication No. 2003-52842 (published on Feb. 25, 2003)

### Non Patent Literature

NPL 1: Kuniyuki Morimoto and six others, “Photodynamic Therapy Using Systemic Administration of 5-Aminolevulinic Acid and a 410-nm Wavelength Light-Emitting Diode for Methicillin-Resistant Staphylococcus aureus-Infected Ulcers in Mice”, PLOS ONE, August 2014, Volume 9, Issue 8 e105173 (published on Aug. 20, 2014)

NPL 2: Makoto Kimura, “Photodynamic Therapy”, Technology Periodical of USHIO INC. “Light Edge”, NO. 38, <Special edition Vol. 3>, (published on October, 2012)

### SUMMARY OF INVENTION

### Technical Problem

However, the conventional techniques described above have following problems. For example, PTL 1 does not specifically disclose how an optimum range of light intensity distribution is realized during treatment or what device is used. It is considered to be essential for a user to correctly set the light intensity distribution. The technique disclosed in PTL 1 has a problem that there is a possibility that human cells are damaged or no treatment is applied depending on radiation conditions because a method of realizing an optimum range of the light intensity distribution during PDT is not disclosed.

Next, PTL 2 discloses a technique by which light emitted from the individual light source units is able to be uniformly radiated, but does not disclose how an optimum range of the light intensity distribution is realized during PDT in a whole of the plurality of light source units. Thus, there is a problem that there is a possibility that human cells are damaged or no treatment is applied depending on radiation conditions.

Next, NPL 2 introduces various PDT devices, but all of them have the two problems described above.

The invention has been made in view of the conventional problems described above, and an object thereof is to provide a photodynamic therapy device capable of improving safety by realizing an optimum range of light intensity distribution during treatment.

### Solution to Problem

In order to solve the aforementioned problems, a photodynamic therapy device according to an aspect of the invention includes: a light source unit including a plurality of light emission elements that emit light having a light emission peak at a specific wavelength; a light detection unit that detects intensity of light emitted by the plurality of light emission elements as light intensity distribution of light emitted by the light source unit; and a light intensity distribution decision unit that decides current, by which each of the plurality of light emission elements is driven, such that the intensity of the light emitted by each of the plurality of light emission elements, which is detected by the light detection unit, falls within a predetermined range.

### Advantageous Effects of Invention

According to an aspect of the invention, an effect of enabling improvement in safety by realizing an optimum range of light intensity distribution during treatment is exerted.

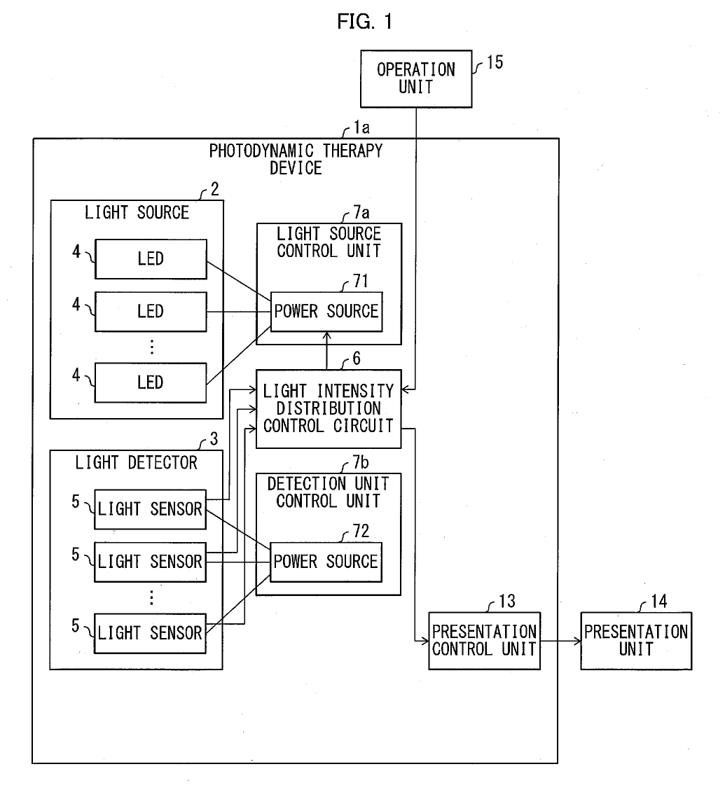

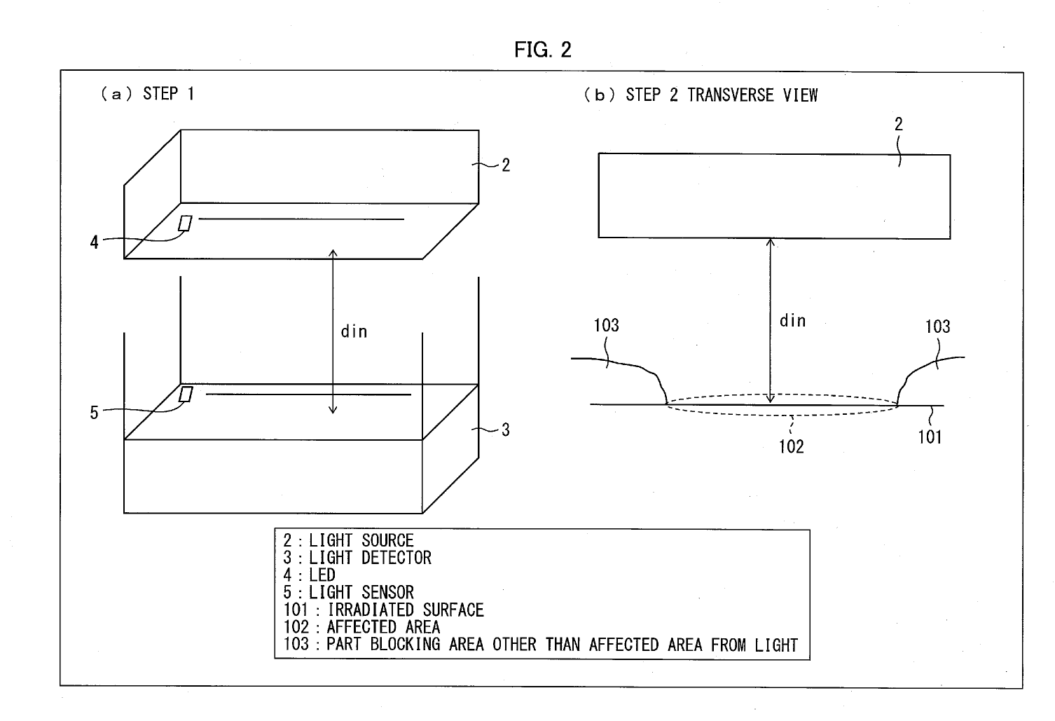

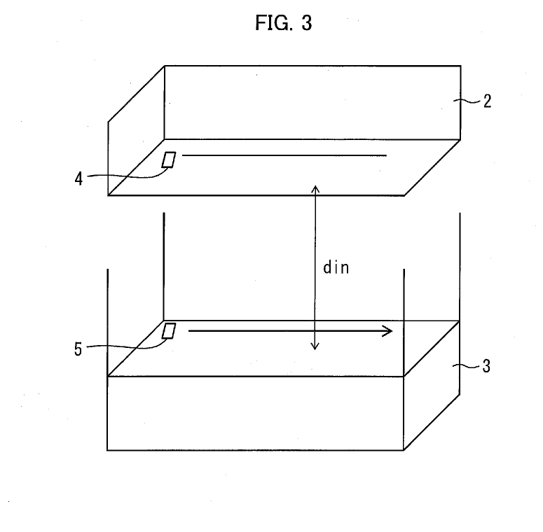

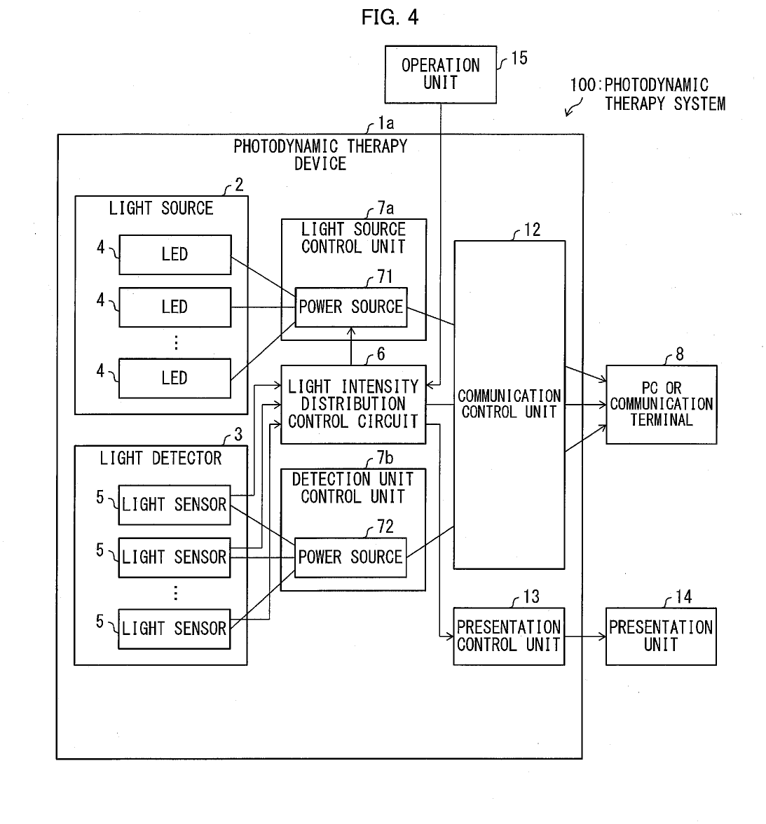

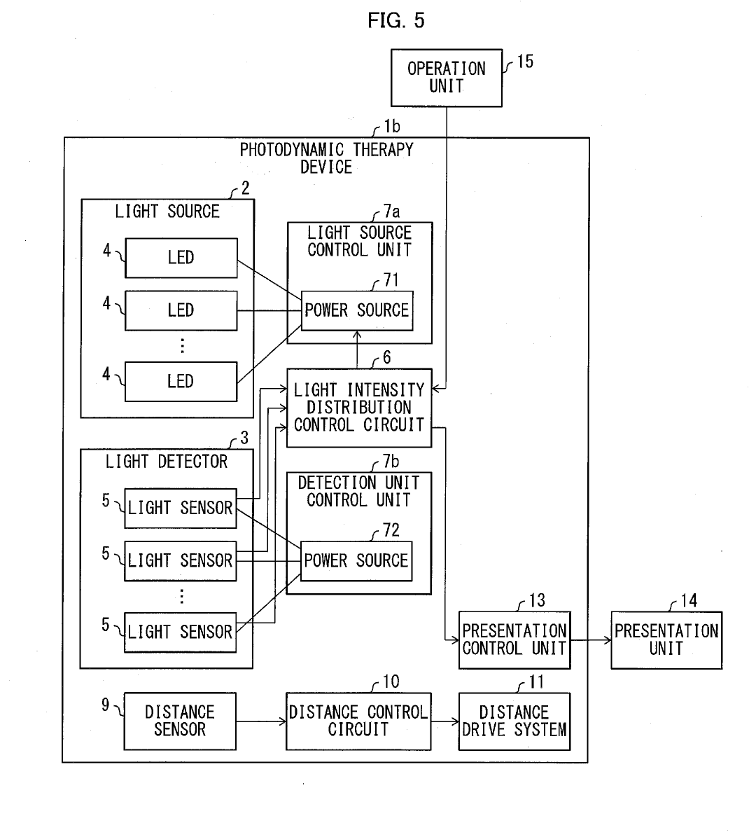

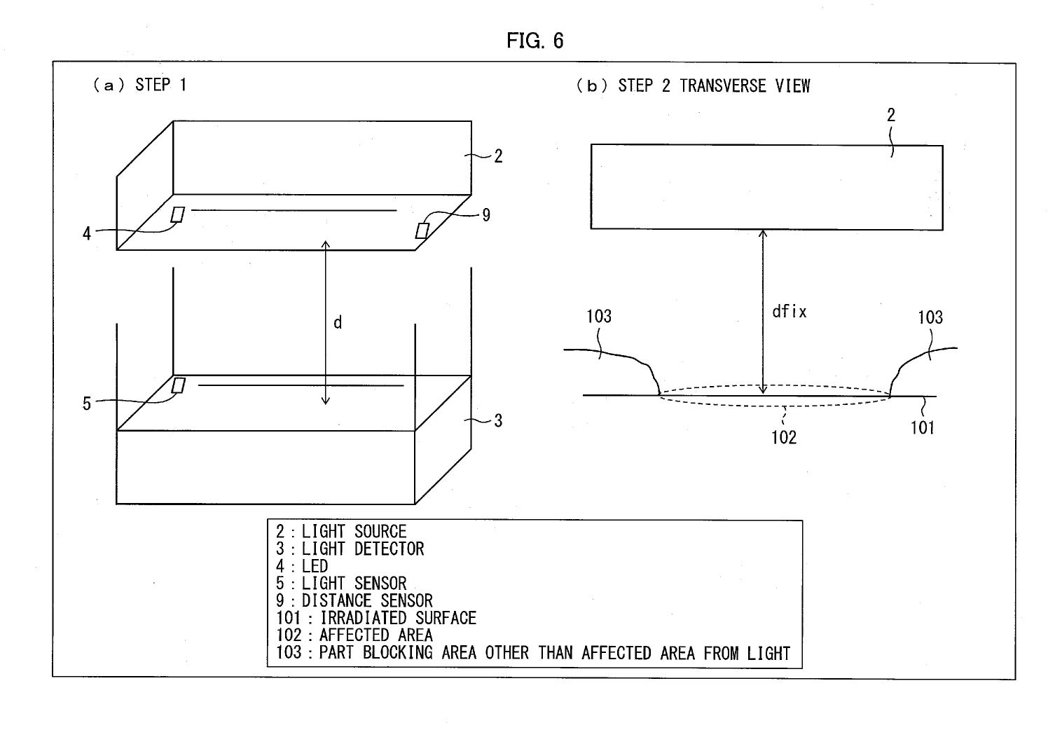

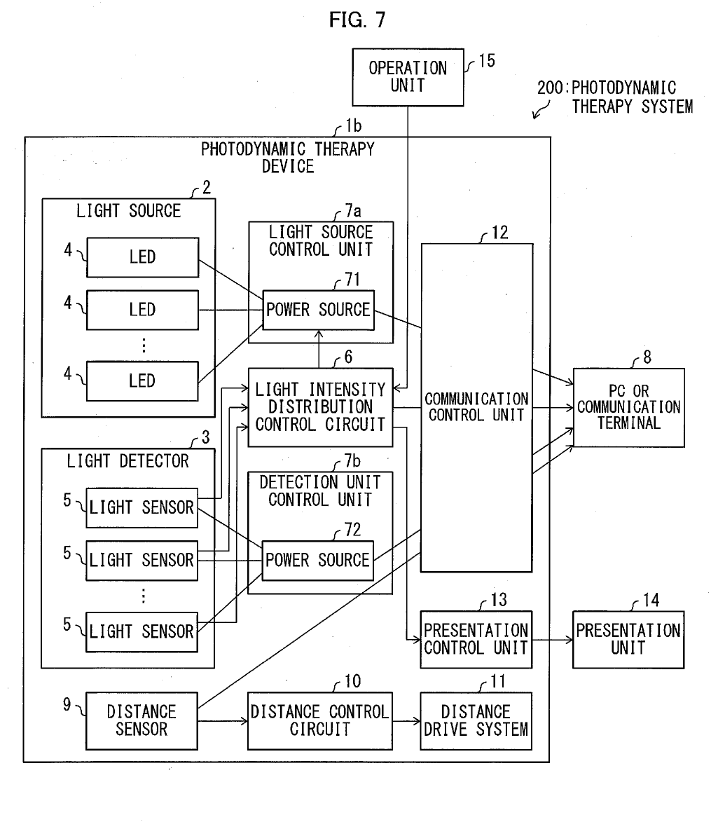

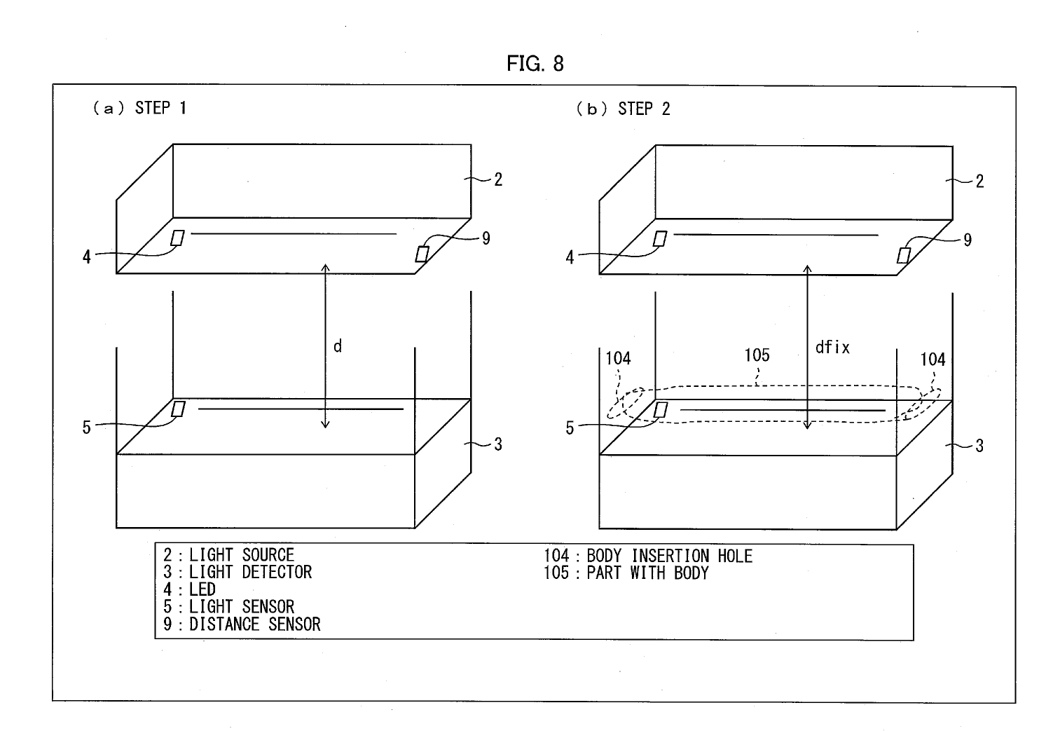

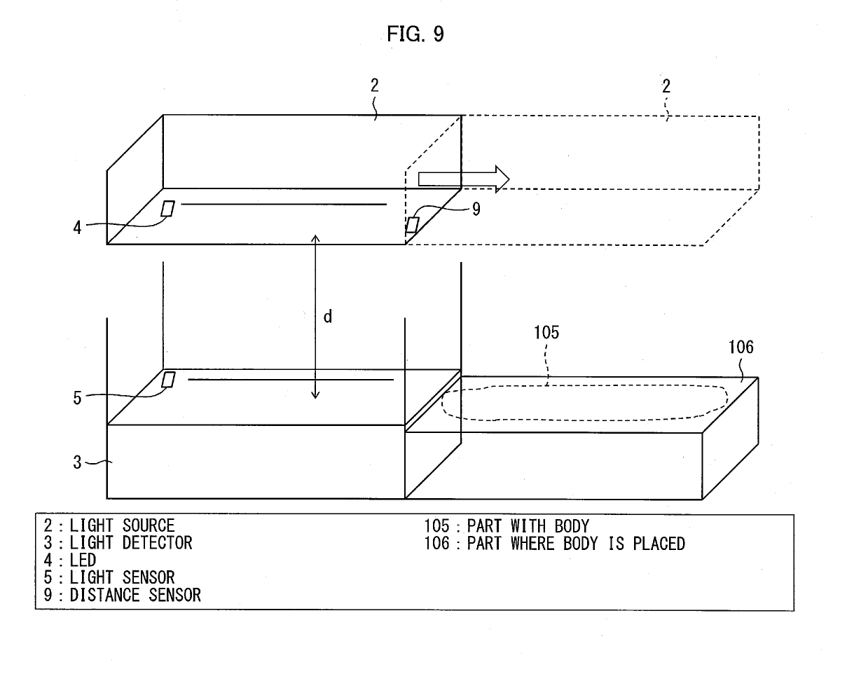

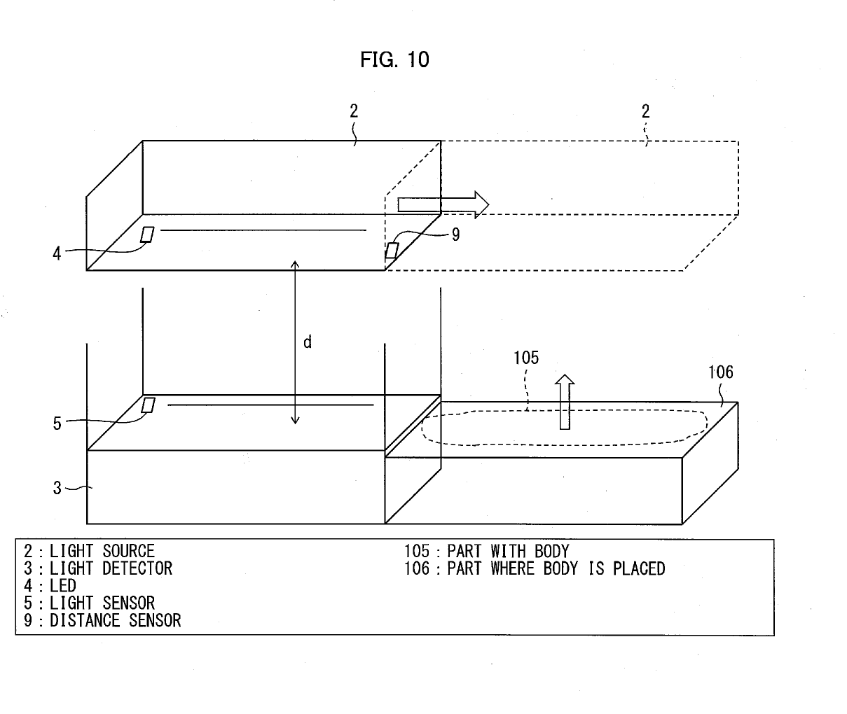

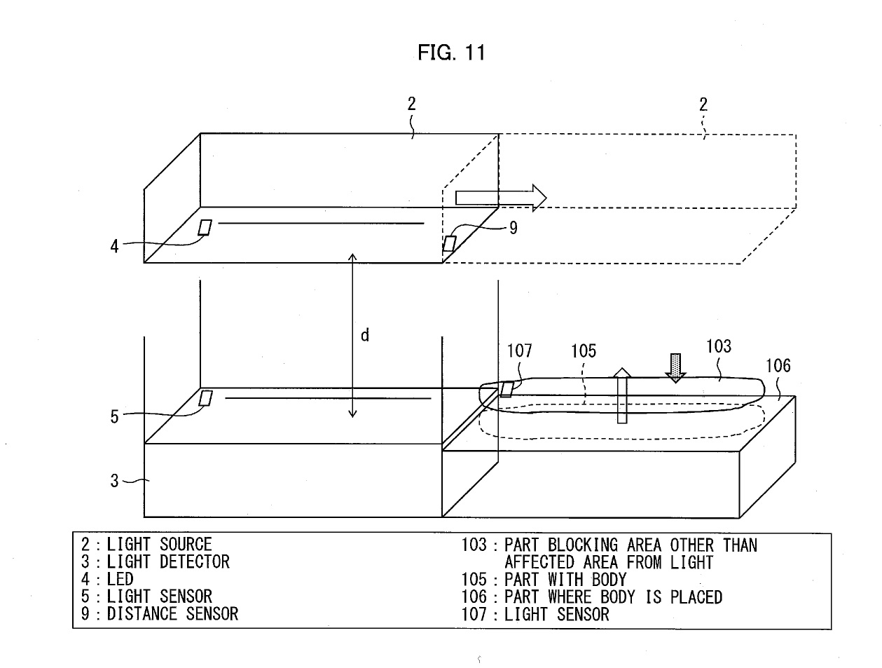

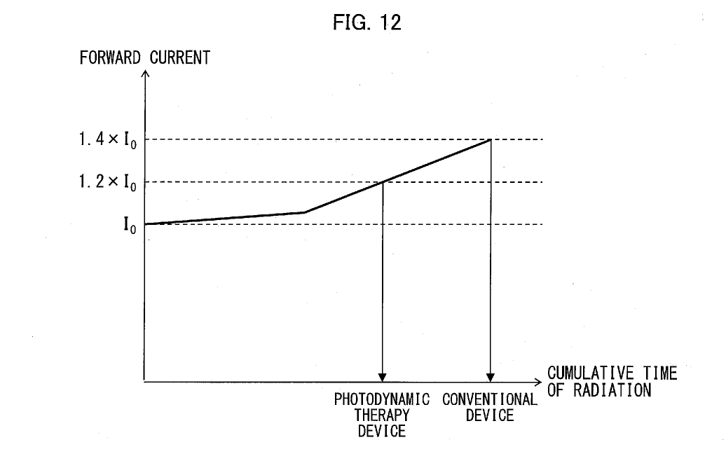

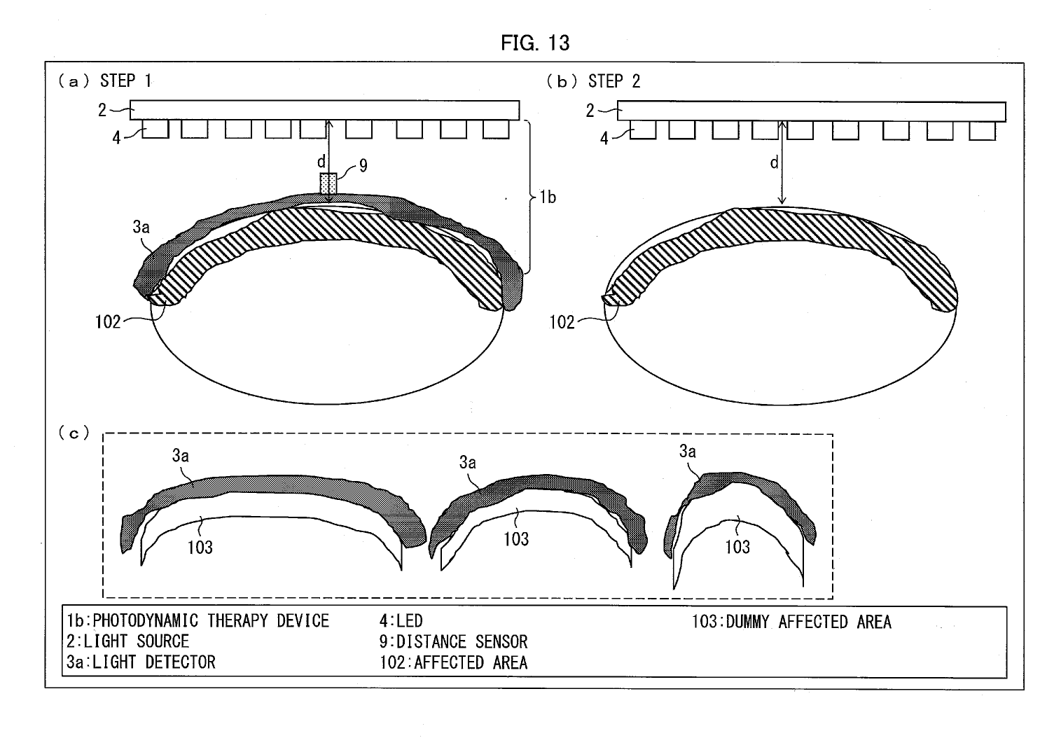

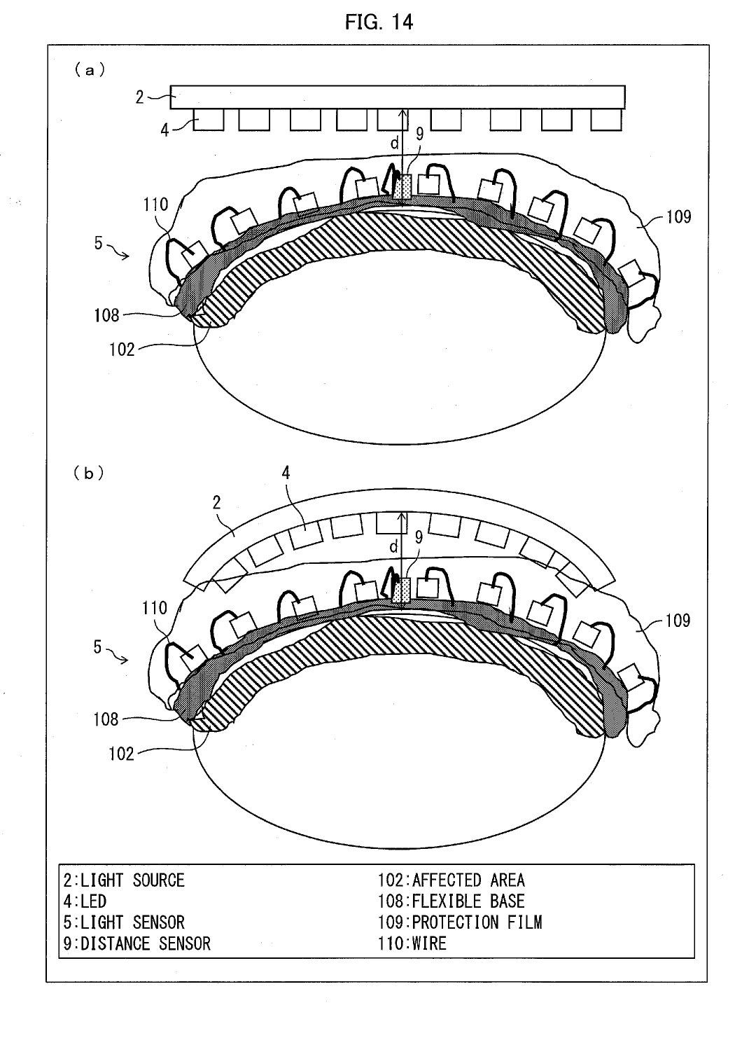

### BRIEF DESCRIPTION OF DRAWINGS

[FIG. 1] FIG. 1 is a block diagram illustrating a configuration of a photodynamic therapy device according to Embodiment 1 of the invention.

[FIG. 2] FIG. 2(a) is a perspective view illustrating a configuration of an appearance of the photodynamic therapy device according to Embodiment 1 above and FIG. 2(b) illustrates a transverse cross section of the photodynamic therapy device according to Embodiment 1 above.

[FIG. 3] FIG. 3 is a perspective view illustrating a configuration of an appearance of a modified example of a light detection unit of the photodynamic therapy device.

[FIG. 4] FIG. 4 is a block diagram illustrating a configuration of a photodynamic therapy system according to Embodiment 2 of the invention.

[FIG. 5] FIG. 5 is a block diagram illustrating a configuration of a photodynamic therapy device according to Embodiment 3 of the invention.

[FIG. 6] FIG. 6(a) is a perspective view illustrating a configuration of an appearance of the photodynamic therapy device according to Embodiment 3 above and FIG. 6(b) illustrates a transverse cross section of the photodynamic therapy device according to Embodiment 3 above.

[FIG. 7] FIG. 7 is a block diagram illustrating a configuration of a photodynamic therapy system according to Embodiment 4 of the invention.

[FIG. 8] FIG. 8 is a schematic view illustrating an example of a method of using the photodynamic therapy device (or the photodynamic therapy system) according to each of Embodiments 1 to 4 above in Embodiment 5 of the invention.

[FIG. 9] FIG. 9 is a schematic view illustrating another example of a method of using the photodynamic therapy device (or the photodynamic therapy system) according to each of Embodiments 1 to 4 above in Embodiment 6 of the invention.

[FIG. 10] FIG. 10 is a schematic view illustrating still another example of a method of using the photodynamic therapy device (or the photodynamic therapy system) according to each of Embodiments 1 to 4 above in Embodiment 7 of the invention.

[FIG. 11] FIG. 11 is a schematic view illustrating still another example of a method of using the photodynamic therapy device (or the photodynamic therapy system) according to each of Embodiments 1 to 4 above in Embodiment 8 of the invention.

[FIG. 12] FIG. 12 is a graph indicating a relation between a cumulative time of radiation and forward current for explaining an advantage obtained by transmitting, before a failure, measurement data or the like to an external communication apparatus in the photodynamic therapy system according to Embodiment 2 or 4 above in Embodiment 9 of the invention.

[FIG. 13] FIG. 13 is a schematic view illustrating an example of a method of using a photodynamic therapy device (or a photodynamic therapy system) according to Embodiment 10 of the invention.

[FIG. 14] FIG. 14 is a schematic view illustrating an example of a method of using a photodynamic therapy device (or a photodynamic therapy system) according to Embodiment 11 of the invention.

### DESCRIPTION OF EMBODIMENTS

Embodiments of the invention will be described as follows with reference to FIGS. 1 to 14. Hereinafter, for convenience of description, the same reference sign is given to a configuration having the same function as described in a certain embodiment, and description thereof may be omitted.

### Embodiment 1

With reference to FIG. 1, a configuration of a photodynamic therapy device la according to Embodiment 1 of the invention will be described. FIG. 1 is a block diagram illustrating the configuration of the photodynamic therapy device 1a. As illustrated in the same figure, the photodynamic therapy device la includes a light source (light source unit) 2, a light detector (light detection unit) 3, a light intensity distribution control circuit (light intensity distribution decision unit) 6, a light source control unit 7a, and a detection unit control unit 7b. A presentation control unit 13 of the photodynamic therapy device la is connected to an external presentation unit 14 and the light intensity distribution control circuit 6 is connected to an external operation unit 15.

### (Light Source 2)

The light source 2 includes a plurality of, for example, ten or more LEDs (light emission elements) 4 to allow measurement of light intensity distribution (or light intensity density distribution). The LEDs 4 of the present embodiment are arranged in a matrix manner (two-dimensional manner). Each of the LEDs 4 emits light with a specific wavelength, for example, in a range of 400 nm to 420 nm as a light emission peak. Note that, the light of the LED 4 may be uniformly radiated light by using, for example, a combination of a convex lens and a concave lens, but a mode for realizing the invention is not limited to such a mode.

### (Light Detector 3)

The light detector 3 includes a plurality of, for example, ten or more light sensors 5. The number of the LEDs 4 and the number of the light sensors 5 do not need to be the same. Each of the light sensors 5 is only required to be sensitive at a specific wavelength ranging from 400 nm to 420 nm emitted by each of the LEDs 4. Imaging by a CCD (Charge Coupling Device) or a CMOS (Complementary Metal-Oxide Semiconductor) may be used instead of Arranging the Light Sensors 5.

### (Light Intensity Distribution Control Circuit 6)

The light intensity distribution control circuit 6 decides current (value), by which each of the plurality of LEDs 4 is driven, so that intensity of the light emitted by each of the plurality of LEDs 4, which is detected by the light detector 3, falls within a predetermined range, and provides the light source control unit 7a with a result of the decision.

### (Power Source 71, Light Source Control Unit 7a)

A power source 71 is electrically connected to each of the LEDs 4 forming the light source 2 and supplies current by which the LED 4 is driven. The light source control unit 7a controls, in accordance with the decision result received from the light intensity distribution control circuit 6, the current value of the current supplied to the LED 4.

More specifically, each of the light from the plurality of LEDs 4 is incident on each of the light sensors 5, and when there is light intensity below a lower limit among pieces of the light intensity that are detected (measured), with the use of the power source 71 via the light intensity distribution control circuit 6, feedback is performed so that the pieces of the light intensity detected by the light sensors 5 reach the lower limit by increasing the current value of the current supplied to each of the LEDs 4. Similarly, when there is light intensity exceeding an upper limit among the pieces of the light intensity that are measured by the light sensors 5, with the use of the power source 71 via the light intensity distribution control circuit 6, feedback is performed so that the pieces of the light intensity by the light sensors 5 reach the upper limit by decreasing the current value of the current supplied to each of the LEDs 4. Note that, the upper limit and the lower limit may be set by a user via the operation unit 15.

When there is light intensity below the lower limit among the pieces of the light intensity that are detected (measured) by the light sensors 5, the presentation control unit 13 may cause the presentation unit 14 to present screen display of “light is too weak” or the like or to produce warning sound. When there is light intensity exceeding the upper limit among the pieces of the light intensity that are detected (measured) by the light sensors 5, the presentation control unit 13 may cause the presentation unit 14 to present screen display of “light is too strong” or the like or to produce warning sound. The presentation unit 14 is constituted by, for example, a display unit (display), a speaker, or the like. With such functions, the light intensity distribution that falls within the predetermined range (within a set range) is able to be obtained.

Note that, though the light intensity (whose unit is mW) is used in the description above, light intensity density (whose unit is mW/cm²) may be used. The light intensity density is able to be easily calculated by dividing the light intensity by an area of the light sensor 5. The light intensity distribution control circuit 6 may have a function for converting the light intensity into the light intensity density.

### (Power Source 72, Detection Unit Control Unit 7b)

A power source 72 is electrically connected to each of the light sensors 5 forming the light detector 3 and supplies current by which the light sensor 5 is driven. The detection unit control unit 7b controls a current value of the current supplied to the light sensor 5. The detection unit control unit 7b also performs control to provide the light intensity distribution control circuit 6 with information about the light intensity (or the light density) detected by the light sensor 5.

The detection unit control unit (determination unit) 7b may be configured to determine, on the basis of the value of the current by which the light detector 3 (the light sensors 5) is driven, whether or not the photodynamic therapy device 1a (or the LEDs 4, the light sensors 5) needs to be replaced. Thereby, the photodynamic therapy device la (or the LEDs 4, the light sensors 5) is able to be replaced at appropriate timing.

An operation of the photodynamic therapy device 1a will be described below. The photodynamic therapy device 1a operates to execute the following steps.

<<Step 1; Decision of photodynamic therapy conditions (referred to as step 1 similarly in the following embodiments)>>

FIG. 2(a) is a view for explaining a method of deciding photodynamic therapy conditions. First, a distance between the light source 2 and the light detector 3 is fixed (the distance is set as din). Then, current is supplied to the LEDs 4 so that the light source 2 is lit.

As described above, the light from the light source 2 is incident on each of the light sensors 5, and when there is light intensity below the lower limit (which may be set by a user) among pieces of the light intensity that are measured, with the use of the power source 71 via the light intensity distribution control circuit 6, feedback is performed so that the pieces of the light intensity by the light sensors 5 reach the lower limit (which may be set by the user) by increasing the current supplied to the LEDs 4. Similarly, when there is light intensity exceeding the upper limit (which may be set by the user) among the pieces of the light intensity measured by the light sensors 5, with the use of the power source 71 via the light intensity distribution control circuit 6, feedback is performed so that the pieces of the light intensity by the light sensors 5 reach the upper limit by decreasing the current supplied to the LEDs 4. With such functions, the light intensity distribution that falls within the set range is able to be obtained.

Meanwhile, the light intensity density is important in the photodynamic therapy in terms of a side effect, and energy density (whose unit is J/cm²) is also important. The required energy density varies in accordance with a type of the PDT, for example, such as a type, concentration, wavelength, or the like of a photosensitive substance to be used. The light source 2 is lit, the light intensity density is measured by the light sensors 5, for example, every one second, and

[Math 1]

Energy density J=∫Eds formula (1)

is obtained. In the formula, E represents the energy density per unit time, and s makes it possible to change the light intensity density E in a stepwise manner or in a pulse form on the basis of a relation of time. The detection unit control unit 7b may have a function for calculating the energy density on the basis of a detection result of the light detector 3. In this case, it may be configured so that the calculation result is provided to the light intensity distribution control circuit 6. At this time, the light intensity distribution control circuit 6 may decide the value of the current supplied to the LEDs 4 so that the energy density falls within a predetermined range.

Note that, the presentation control unit 13 illustrated in FIG. 1 may perform control to cause the presentation unit 14 to perform screen display of, for example, the current supplied to the LEDs 4 before and after the feedback, data related to the light intensity, the light intensity distribution, the light intensity density, or the light intensity density distribution each of which is measured by the light sensors 5, or an image thereof obtained through imaging. The presentation control unit 13 may be configured to perform control so that the presentation unit 14 performs screen display of a cumulative time of radiation (time during which the light source 2 is lit) or the like or produces warning sound or the like.

>>Step 2; Photodynamic therapy (referred to as step 2 similarly in the following embodiments)>>

Next, FIG. 2(b) is a transverse sectional view of the photodynamic therapy device la that conducts photodynamic therapy. An affected area is irradiated with light under the radiation conditions (such as the current supplied to the LEDs 4, a distance between the light source 2 and the affected area, and radiation time) that are decided in advance by step 1 above. The photodynamic therapy that does not use local radiation by laser is desired to be conducted while an area other than an affected area 102 that is desired to be irradiated with light (i.e. desired to be treated) is blocked from light as illustrated in FIG. 2(b) (refer to a part blocking an area other than an affected area from light 103). The reason therefor is considered as follows, for example: heat from the light source 2 is minimized; or a site in which photosensitivity develops is minimized.

<<Effect of Photodynamic Therapy Device 1a>>

According to the aforementioned embodiment, the current by which each of the plurality of LEDs 4 is driven is decided so that the intensity of the light emitted from each of the plurality of LEDs 4 falls within the predetermined range. Thus, when the light intensity of the LEDs 4 falls within an appropriate range, an optimum range of the light intensity distribution is able to be realized during treatment. This makes it possible to improve safety of the photodynamic therapy device la. As a result, with the photodynamic therapy device la, safety is able to be improved by realizing the optimum range of the light intensity distribution during treatment.

### <<About Modified Example of Light Detection Method>>

Though a mode in which the plurality of light sensors 5 are arranged in a matrix manner (two-dimensional manner) has been described as the mode of the light detector 3 in the aforementioned embodiment, but a mode for realizing the invention is not limited thereto. For example, as illustrated in FIG. 3, a configuration in which one (or more) light sensor 5 is used to perform scanning and the intensity of the light output from each of the LEDs 4 is detected chronologically may be adopted.

### Embodiment 2

Next, with reference to FIG. 4, a configuration of a photodynamic therapy system 100 according to Embodiment 2 of the invention will be described. FIG. 4 is a block diagram illustrating the configuration of the photodynamic therapy system 100.

A difference from the mode illustrated in FIG. 1 lies in that the photodynamic therapy device la includes a communication control unit (transmission control unit) 12 and is able to communicate with an external PC or communication terminal (communication apparatus) 8 via the communication control unit 12 in the photodynamic therapy system 100 of the present embodiment.

### (Communication Control Unit 12)

The communication control unit 12 may be configured to perform control so that information about the value of the current by which each of the plurality of LEDs 4 is driven is transmitted to the external PC or communication terminal 8. As a result, by performing data communication with the information about the value of the current by which each of the plurality of LEDs 4 is driven, it is possible to realize prevention of a failure, immediate maintenance, or immediate replacement.

The communication control unit 12 may be configured to perform control so that information (which may be light intensity distribution or light intensity density distribution) about the intensity of the light emitted by each of the plurality of LEDs 4, which is detected by the light detector 3 (the light sensors 5), is transmitted to the PC or communication terminal 8. As a result, by performing data communication with the information about the intensity of the light emitted by each of the plurality of LEDs 4, it is possible to realize prevention of a failure, immediate maintenance, or immediate replacement.

The communication control unit 12 may transmit information about the value of the current by which the light detector 3 (the light sensors 5) is driven to the PC or communication terminal 8. As a result, by performing data communication with the information about the value of the current by which the light detector 3 (the light sensors 5) is driven, it is possible to realize prevention of a failure, immediate maintenance, or immediate replacement.

When the light intensity distribution control circuit 6 determines that the light intensity density, the light intensity density distribution, or the like does not fall within a prescribed range, the communication control unit 12 may transmit information about warning thereof to the PC or communication terminal 8.

An operation of the photodynamic therapy system 100 will be described below. The photodynamic therapy system 100 operates to execute the following steps.

At step 1 above, the communication control unit 12 performs control so that information about the current which is supplied to the LEDs 4 before and after the control, the light intensity, the light intensity distribution, the light intensity density, the light intensity density distribution, or the like each of which is measured by the light sensors 5 is transmitted by the PC or communication terminal 8.

At step 2 above, the communication control unit 12 performs control so that information about the current supplied to the LEDs 4, a radiation time, a cumulative time of radiation, or the like is transmitted to the PC or communication terminal 8.

### <<Effect of Photodynamic Therapy System 100>>

According to the photodynamic therapy system 100 of the present embodiment, the following three effects are expected.

(1) A state of use of the photodynamic therapy device la is able to be known without a visit to or contact with a user.
(2) Maintenance timing or replacement timing of the photodynamic therapy device 1a is able to be known without a visit to or contact with a user.
(3) Since a failure of the photodynamic therapy device la is able to be prevented from occurring, it is less likely that the photodynamic therapy device la is not able to be used when it is required.

Though sales representatives need to be arranged for individual users or areas to perform maintenance in conventional photodynamic therapy devices, the three effects make it possible to perform the maintenance by a host computer and the less number of sales representative as compared with the conventional one and achieve cost reduction.

### Embodiment 3

Next, a configuration of a photodynamic therapy device 1b according to Embodiment 3 of the invention will be described with reference to FIG. 5. FIG. 5 is a block diagram illustrating a configuration of the photodynamic therapy device 1b.

A difference from the aforementioned mode lies in that the photodynamic therapy device lb of the present embodiment includes a distance sensor 9, a distance control circuit (distance determination unit) 10, and a distance drive system (drive unit) 11.

### (Distance Sensor 9)

The distance sensor 9 is configured to detect a distance between the light source 2 and the light detector 3. The distance control circuit 10 is configured to determine whether or not the distance detected by the distance sensor 9 falls within a predetermined range. The distance drive system 11 is configured to perform control to change the distance between the light source 2 and the light detector 3 to fall within the predetermined range when the distance control circuit 10 determines that the distance does not fall within the predetermined range. The light intensity distribution of the light source 2 varies in accordance with the distance between the light source 2 and the light detector 3 in many cases. When heat is generated from the light source 2 in the photodynamic therapy, a photosensitive substance may be deteriorated or the heat may be painful to a patient. Thus, it is desired as in the aforementioned configuration that the control is performed so that the distance between the light source 2 and the light detector 3 falls within the predetermined range. That is, at least at step 2 above, a mechanism of making a radiation distance constant or changed is desired to be provided as in the present embodiment. In response to such a demand, the photodynamic therapy device lb is obtained by adding the distance sensor 9, the distance control circuit 10, and the distance drive system 11 to the photodynamic therapy device 1a described above.

An operation of the photodynamic therapy device lb will be described below. The photodynamic therapy device 1b operates to execute the following steps.

For example, as illustrated in FIG. 6(a), at step 1, when the distance (distance d) between the light source 2 and the light detector 3 is detected by the distance sensor 9 and the distance is too close to a distance lower limit that is set in advance, the distance drive system 11 is moved through the distance control circuit 10 to extend the distance to the light source 2 or the light detector 3. Note that, when the distance is too close to the distance lower limit, the presentation control unit 13 may be configured to cause the presentation unit 14 to perform screen display of “light source is too close” or the like or to produce warning sound.

When the distance is too far from a distance upper limit that is set in advance, the distance is able to be made closer in the same manner. Note that, when the distance is too far from the distance upper limit, the presentation control unit 13 may be configured to cause the presentation unit 14 to perform screen display of “light source is too far” or the like or to produce warning sound. As described above, the distance control circuit 10 may decide an appropriate distance dfix. The presentation control unit 13 may perform control to cause the presentation unit 14 to perform screen display of the distance decided as described above.

Next, for example, as illustrated in FIG. 6(b), at step 2, a distance between the affected area 102 and the light source 2 is subjected to feedback in the same manner and corrected to the appropriate distance dfix. The correction to the appropriate distance may be performed by a manual operation.

### Embodiment 4

Next, with reference to FIG. 7, a configuration of a photodynamic therapy system 200 according to Embodiment 4 of the invention will be described. FIG. 7 is a block diagram illustrating the configuration of the photodynamic therapy system 200.

A difference from the mode illustrated in FIG. 5 lies in that the photodynamic therapy device lb includes the communication control unit (transmission control unit) 12 and is able to communicate with the external PC or communication terminal (communication apparatus) 8 via the communication control unit 12 in the photodynamic therapy system 200 of the present embodiment.

### (Communication Control Unit 12)

A state where the distance is farther from the upper limit that is set in advance when control for the current supplied to the LEDs 4 and distance control are performed at step 1 of Embodiment 3 means a state where the light source 2 is deteriorated over time. Thus, the communication control unit 12 may transmit the distance decided through the distance control and information about associated warning, which have been described in Embodiment 3, to the PC or communication terminal 8.

### Embodiment 5

### Application Example 1 of Embodiments 1 to 4

Regarding Embodiments 1 to 4 described above, when a body insertion hole 104 is provided substantially in parallel to the light source 2, for example, as illustrated in FIG. 8(b), a part with a body 105 is able to be uniformly irradiated with light from the light source 2. In the present embodiment, the steps are as follows. Note that, though the following description will be given with respect to Embodiment 4 described above, the similar is also applied to Embodiments 1 to 3 described above. Step 1 is similar to that of Embodiment 3, so that description thereof will be omitted.

At step 2, as illustrated in FIG. 8(b), for example, the light source 2 and the light detector 3 are held so that a distance therebetween is the same as the distance between the light source 2 and the light detector 3 that is decided at step 1. A body is inserted in the part with a body 105 through the body insertion hole 104. The body insertion hole 104 has a mechanism of supporting a part of the inserted body and is able to fix the part of the body. This makes it possible to perform radiation under conditions closer to the radiation conditions decided at step 1. With the light sensor 5 that is not hidden by the part of the body, monitoring of the intensity of the light from the light source 2 is able to be performed. Thereby, various side effects caused by a small effect of the photodynamic therapy or strong light are able to be prevented.

### Embodiment 6

### Application Example 2 of Embodiments 1 to 4

Regarding Embodiments 1 to 4 described above, in the present embodiment, as illustrated in FIG. 9, for example, a part where a body is placed 106 is further provided when the light source 2 relatively moves (slides) with respect to a position at which the light detector 3 is disposed. Step 1 is similar to that of Embodiment 3, so that description thereof will be omitted.

At step 2, an operation is able to be performed as follows so as to realize the radiation conditions decided at step 1, for example.

(1) A part of the body that is desired to be subjected to the photodynamic therapy is held on the part where a body is placed 106 (a fixing belt may be provided).
(2) The light source 2 is lit under the radiation conditions decided at step 1.

### Embodiment 7

### Application Example 3 of Embodiments 1 to 4

Regarding Embodiments 1 to 4 described above, in the present embodiment, as illustrated in FIG. 10, for example, the part where a body is placed 106 is further provided when the light source 2 relatively moves (slides) with respect to a position at which the light detector 3 is disposed. In the present embodiment, the mechanism of moving the part where a body is placed 106 may be provided. For example, thickness of the body is measured in advance and the part where a body is placed 106 is vertically moved by the thickness (finally to a position lower than a position at which the light sensor 5 is disposed). Step 1 is similar to that of Embodiment 3, so that description thereof will be omitted.

At step 2, an operation is able to be performed as follows so as to realize the radiation conditions decided at step 1, for example.

(1) A part of the body that is desired to be subjected to the photodynamic therapy is held on the part where a body is placed 106 (a fixing belt may be provided).
(2) Thickness of the part of the body is measured.
(3) The part where a body is placed 106 is moved to be away from the light source 2 by the thickness measured in (2) above.
(4) The light source 2 is lit under the radiation conditions decided at step 1.

### Embodiment 8

### Application Example 4 of Embodiments 1 to 4

Regarding Embodiments 1 to 4 described above, in the present embodiment, as illustrated in FIG. 11, for example, the part where a body is placed 106 is further provided when the light source 2 relatively moves (slides) with respect to a position at which the light detector 3 is disposed. In the present embodiment, the mechanism of moving the part where a body is placed 106 is provided. In the present embodiment, by further providing a part blocking an area other than an affected area from light 103, to which a light sensor 107 is attached, real-time monitoring of the intensity of the light radiated to the affected area may be performed. For example, it may be configured so that the light sensor 107 is attached to a cloth blocking an area other than an affected area from light 103, and when the detected light intensity is equal to or greater than a prescribed value, the current is shut off. This makes it possible to prevent a trouble due to excessive radiation.

It is also possible to control the current, which is supplied to the LEDs 4, with the light intensity measured by the light sensor 107 to change the light intensity of the light source 2. Thereby, various side effects caused by a small effect of the photodynamic therapy or strong light are able to be prevented.

### Embodiment 9

Regarding Embodiments 1 to 4 described above, in the present embodiment, when it is determined that forward current IF applied to the LEDs 4 reaches a certain value (for example, 1.2 times of an initial value, refer to FIG. 12) as a result of the feedback, the detection unit control unit (determination unit) 7b of each of the photodynamic therapy devices 1a and 1b described above may notify the presentation control unit 13 of the determination. At this time, the presentation control unit 13 may be configured to perform control to cause the presentation unit 14 to present an alert (warning).

As above, though maintenance has been conventionally performed in the case of a failure (I=1.4×I₀), a preliminary point of 1.2×I₀ is set in advance. As a result, by performing maintenance or replacement at the time point of I=1.2×I₀, an unavailable period is able to be minimized.

Note that, the 1.2 times may be allowed to be set by a user via the operation unit 15. As a result, though maintenance or replacement has been conventionally considered in the case of a failure (for example, 1.4 times of an initial value, refer to FIG. 12) and inconvenience has occurred in usage of the photodynamic therapy device in some cases, by including a function for predicting failure timing in advance, the inconvenience in usage is minimized. Needless to say, it is more desirable to further include the communication function described in Embodiment 2 or 4.

### Embodiment 10

Next, an operation of the photodynamic therapy device lb according to Embodiment 10 of the invention will be described with reference to FIG. 13. FIG. 5 is a block diagram illustrating the configuration of the photodynamic therapy device 1b. The photodynamic therapy device lb of the present embodiment is different from that of the mode described above in that the light detector 3a is able to change a shape thereof along a shape of an affected area (for example, the light detector 3a is bent along the affected area 102).

The PDT (Photo Dynamic Therapy) is conducted for an affected area that is curved, for example, such as an arm, a face, or a buttock portion in many cases. Only when the shape of the light detector 3a changes (for example, is curved) along the shape of the affected area, the light intensity distribution according to the shape of the affected area is able to be measured accurately. Thereby, it is possible for the first time to realize the accurate light intensity distribution also for the affected area that is curved.

An operation of the photodynamic therapy device lb will be described below. The photodynamic therapy device lb operates to execute the following steps. For example, as illustrated in FIG. 13(a), at step 1, first, the light detector 3a surrounds (or may be attached to with tape or the like) the affected area 102, and the light detector 3a having a curvature according to the affected area 102 is selected. When it is difficult to do so, for example, because the affected area 102 is painful, a dummy affected area 103 of FIG. 13(c) having a curvature close to that of the affected area as illustrated in FIG. 13(c) is prepared in advance and the light detector 3a having the corresponding curvature is selected.

The light detector 3a may be constituted by, for example, a curved CMOS or CCD, or resin whose color changes in accordance with the light intensity. Any light detector 3a is able to be used as long as being able to detect (indicate) the light intensity. With the use of the distance sensor 9, a distance between the light source 2 and the light detector 3a is adjusted to an appropriate distance. The light source 2 is lit by applying current to each of the LEDs 4. When the light detector 3a has the same shape as that of the affected area 102, the intensity distribution of the light that the affected area 102 actually receives is able to be measured. The current applied to each of the LEDs 4 is controlled so that the light intensity distribution or the light intensity, which is measured by the light detector 3a, falls within a value range that is set in advance.

Then, as illustrated in FIG. 13(b), at step 2, the light detector 3a is detached from the affected area 102. This operation is not performed when the dummy affected area is used. The light source 2 is lit by applying current to each of the LEDs 4. Thereby, uniform light intensity distribution is able to be obtained even for the affected area 102 that does not have a straight shape.

### Embodiment 11

Next, as a modified example of the light detector 3a of Embodiment 10, as illustrated in FIG. 14(a), for example, the light sensor 5 may be arranged on a flexible base 108 and the light sensor 5 and the distance sensor 9 may be connected via a wire 110. That is, the present embodiment is different from the mode described above in that the light detector 3a has a structure in which the light sensor 5 is mounted on the flexible base 108.

With the aforementioned configuration, when the light sensor 5 is mounted on the flexible base 108, it is possible to produce the light detector 3a that is inexpensive and is able to measure accurate light intensity distribution for an affected area that is curved. Note that, a protection film 109 is attached to protect the wire 110. A mode in which the light sensor 5 is mounted on the flexible base 108 is not limited to the illustrated mode.

### Embodiment 12

Next, a modified example of Embodiment 10 described above (photodynamic therapy device of Embodiment 12) is illustrated in FIG. 14(b). The present modified example is different from the mode described above in that the light source 2 is able to change a shape thereof (for example, the light source 2 is able to be curved) along a shape of the affected area 102 in the photodynamic therapy device of Embodiment 10. This makes it possible to perform light radiation in a form according to the affected area 102 and obtain more uniform light intensity distribution.

For example, the light source 2 may have a structure in which the LED 4 is mounted on the flexible base. According to such a configuration, when the light source 2 is also flexible, the light source 2 is able to closely adhere to the affected area at step 2 above. Even when a patient moves, the light intensity distribution measured at step 1 above is able to be always realized.

### Conclusion

A photodynamic therapy device according to an aspect 1 of the invention has a configuration of including: a light source unit (light source 2) including a plurality of light emission elements (LEDs 4) that emit light having a light emission peak at a specific wavelength; a light detection unit (light detector 3) that detects intensity of light emitted by the plurality of light emission elements as light intensity distribution of light emitted by the light source unit; and a light intensity distribution decision unit (light intensity distribution control circuit 6) that decides current, by which each of the plurality of light emission elements is driven, such that the intensity of the light emitted by each of the plurality of light emission elements, which is detected by the light detection unit, falls within a predetermined range.

According to the aforementioned configuration, the current by which each of the plurality of light emission elements is driven is decided so that the intensity of the light emitted by each of the plurality of light emission elements falls within the predetermined range. Thus, when the light intensity of the light emission elements falls within an appropriate range, an optimum range of the light intensity distribution is able to be realized during treatment. This makes it possible to improve safety of the photodynamic therapy device.

Accordingly, with the aforementioned configuration, safety is able to be improved by realizing the optimum range of the light intensity distribution during treatment.

A photodynamic therapy device according to an aspect 2 of the invention may further include, in the aspect 1, a transmission control unit (communication control unit 12) that transmits, to an external communication apparatus, information about a value of the current by which each of the plurality of light emission elements is driven. According to the aforementioned configuration, by performing data communication with the information about the value of the current by which each of the plurality of light emission elements is driven, it is possible to realize prevention of a failure, immediate maintenance, or immediate replacement.

In a photodynamic therapy device according to an aspect 3 of the invention, the transmission control unit may transmit, to the communication apparatus, information about the intensity of the light emitted by each of the plurality of light emission elements, which is detected by the light detection unit, in the aspect 2. According to the aforementioned configuration, by performing data communication with the information about the intensity of the light emitted by each of the plurality of light emission elements, it is possible to realize prevention of a failure, immediate maintenance, or immediate replacement.

In a photodynamic therapy device according to an aspect 4 of the invention, the transmission control unit may transmit, to the communication apparatus, information about a value of current, by which the light detection unit is driven, in the aspect 2 or 3. According to the aforementioned configuration, by performing data communication with the information about the value of the current by which the light detection unit is driven, it is possible to realize prevention of a failure, immediate maintenance, or immediate replacement.

A photodynamic therapy device according to an aspect 5 of the invention may further include in any of the aspects 1 to 4: a distance sensor that detects a distance between the light source unit and the light detection unit; a distance determination unit that determines whether or not the distance detected by the distance sensor falls within a predetermined range; and a drive unit that, when it is determined by the distance determination unit that the distance does not fall within the predetermined range, changes the distance between the light source unit and the light detection unit to fall within the predetermined range.

The light intensity distribution of the light source unit varies in accordance with the distance between the light source unit and the light detection unit in many cases. When heat is generated from the light source unit in the photodynamic therapy, a photosensitive substance may be deteriorated or the heat may be painful to a patient. Thus, it is desired as in the aforementioned configuration that the distance between the light source unit and the light detection unit is able to be changed so as to fall within the predetermined range.

A photodynamic therapy device according to an aspect 6 of the invention may further include, in any of the aspects 1 to 5, a determination unit that determines whether or not replacement of the photodynamic therapy device is necessary on the basis of the value of the current by which the light detection unit is driven. According to the aforementioned configuration, the photodynamic therapy device is able to be replaced at appropriate timing.

In a photodynamic therapy device according to an aspect 7 of the invention, the light detection unit may be allowed to change a shape thereof along a shape of an effected area in any of the aspects 1 to 6.

The PDT (Photo Dynamic Therapy) is conducted for an affected area that is curved, for example, such as an arm, a face, or a buttock portion in many cases. Only when the shape of the light detection unit changes (for example, is curved) along the shape of the affected area, the light intensity distribution according to the shape of the affected area is able to be measured accurately. Thereby, it is possible for the first time to realize the accurate light intensity distribution also for the affected area that is curved.

In a photodynamic therapy device according to an aspect 8 of the invention, the light detection unit may have a structure in which a light sensor is mounted on a flexible base in the aspect 7.

According to the aforementioned configuration, when the light sensor is mounted on the flexible base, it is possible to produce the light detection unit that is inexpensive and is able to measure accurate light intensity distribution for an affected area that is curved.

In a photodynamic therapy device according to an aspect 9 of the invention, the light source unit may have a structure in which the light emission element is mounted on a flexible base in the aspect 7 or 8.

According to the aforementioned configuration, when the light source unit is also flexible, the light source unit is able to closely adhere to the affected area. Even when a patient moves, the light intensity distribution measured is able to be always realized.

### Other Expression of Invention

In a photodynamic therapy device according to an aspect of the invention, the light detection unit may have a variable shape along a shape of an affected area that is curved. The PDT is conducted for an affected area that is curved, for example, such as an arm, a face, or a buttock portion in many cases. Only when the light detection unit is curved, the light intensity distribution according to the shape of the affected area is able to be measured accurately. Thereby, it is possible for the first time to realize the accurate light intensity distribution also for the affected area that is curved.

In a photodynamic therapy device according to another aspect of the invention, the light detection unit may have a light sensor mounted on a flexible base. As a modified example of the light detection unit according to the aspect, various modes of a mode in which an image sensor such as a curved CCD or CMOS is included, a mode in which resin whose color changes in accordance with the light intensity is included, and the like are considered, and when the light sensor is mounted on the flexible base, it is possible to produce the light detection unit that is inexpensive and is able to measure accurate light intensity distribution for an affected area that is curved.

In a photodynamic therapy device according to another aspect of the invention, the light source unit may have an LED mounted on a flexible base. When the light source unit is also flexible, the light source unit is able to closely adhere to the affected area. Even when a patient moves, the light intensity distribution measured is able to be always realized.

### Additional Notes

The invention is not limited to each of the embodiments described above and can be modified variously within the scope defined by the claims, and embodiments obtained by appropriately combining technical means disclosed in different embodiments are also included in the technical scope of the invention. Further, by combining the technical means disclosed in each of the embodiments, a new technical feature may be formed.

### INDUSTRIAL APPLICABILITY

The invention is able to be utilized for a photodynamic therapy device used for a photodynamic therapy, and is particularly suitable for a photodynamic therapy device that minimizes photosensitivity and has excellent utility.

### REFERENCE SIGNS LIST

1a, 1b photodynamic therapy device

2 light source (light source unit)

3 light detector (light detection unit)

4 LED (light emission element)

6 light intensity distribution control circuit (light intensity distribution decision unit)

8 PC or communication terminal (communication apparatus)

9 distance sensor

10 distance control circuit (distance determination unit)

11 distance drive system (drive unit)

12 communication control unit (transmission control unit)

100, 200 photodynamic therapy system

1-9. (canceled)

10. A photodynamic therapy device, comprising:
a plurality of light emission elements;
a detection unit that detects distribution of intensity of light of the plurality of light emission elements; and
decision unit that decides current, by which the plurality of light emission elements are driven, such that the distribution of the light intensity detected by the detection unit falls within a predetermined range, wherein
the decision unit, when there is light intensity lower than a predetermined lower limit in the distribution of the light intensity, controls current, by which a corresponding light emission element among the plurality of light emission elements is driven, such that the light intensity reaches the lower limit, and when there is light intensity higher than a predetermined upper limit in the distribution of the light intensity, controls current, by which a corresponding light emission element among the plurality of light emission elements is driven, such that the light intensity reaches the upper limit.

11. The photodynamic therapy device according to claim 10, further comprising a transmission control unit that transmits, to outside, information about a value of the current by which the plurality of light emission elements are driven.

12. The photodynamic therapy device according to claim 11, wherein
the transmission control unit
transmits, to outside, information about the intensity of the light of the plurality of light emission elements, which is detected by the detection unit.

13. The photodynamic therapy device according to claim 11, wherein the transmission control unit transmits, to outside, information about a value of current by which the detection unit is driven.

14. The photodynamic therapy device according to claim 10, further comprising:
a sensor that measures a distance between each of the plurality of light emission elements and the detection unit;
a distance determination unit that determines whether or not the distance falls within a predetermined range; and
a drive unit that, when it is determined by the distance determination unit that the distance does not fall within the predetermined range, changes the distance to fall within the predetermined range.

15. The photodynamic therapy device according to claim 10, further comprising a determination unit that determines whether or not replacement of the photodynamic therapy device is necessary on a basis of the value of the current by which the detection unit is driven.

16. The photodynamic therapy device according to claim 10, wherein the detection unit is allowed to change a shape thereof along a shape of an effected area.

17. The photodynamic therapy device according to claim 16, wherein the detection unit has a light sensor placed on a flexible base.

18. The photodynamic therapy device according to claim 16, wherein the plurality of light emission elements are placed on a flexible base.
